(19) United States
(12) Patent Application Publication
(10) Pub. No.: US 2009/0197789 A1
(43) Pub. Date: Aug. 6, 2009

(54) ACETYLATION OF CHITOSAN

(76) Inventors: Anju Deepali Massey Brooker, Newcastle/Tyne (GB); Alan Thomas Brooker, Newcastle/Tyne (GB); Katherine Helen Combs, Cincinnati, OH (US); Larry Eugene Miller, Cincinnati, OH (US); Stephen Godfrey, Altrincham (GB); Philip Robert Mallender, Manchester (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/357,451

(22) Filed: Jan. 22, 2009

Related U.S. Application Data
(60) Provisional application No. 61/063,077, filed on Jan. 31, 2008.

(51) Int. Cl. C11D 17/08; C07H 1/00; C07H 3/00

(52) U.S. Cl. 510/296; 536/124; 536/20

(57) ABSTRACT

A process for making modified N-acetylated chitosan comprising the steps of:
a) forming an aqueous solution of chitosan in aqueous acid;
b) mixing the resulting solution of step a) with an acetylating agent in a chitosan:acetylating agent sub-stoichiometric amount; and
c) allowing the chitosan and acetylating agent to react for a length of time sufficient for substantial completion of the acetylation reaction.

### CROSS REFERENCES TO RELATED APPLICATIONS

This application claims priority under 37 U.S.C. §119(e) to U.S. Provisional Application Ser. No. 61/063,077 filed Jan. 31, 2008.

### TECHNICAL FIELD

The present invention relates to a process for acetylating chitosan, especially for N-acetylating chitosan. The invention also relates to detergent products comprising chitosan obtainable according to the process.

### BACKGROUND OF THE INVENTION

Chitin is the main constituent in the shells of crustaceans and is the most abundant naturally occurring biopolymer other than cellulose. Chitosan is derived from chitin and can be formed by deacetylation of chitin. Chitosan is commercially available in a wide variety of molecular weights (e.g., 10-1,000 kDa) and deacetylation degrees. Chitosan is used for a wide variety of purposes including plant care, cosmetics additives, cleaning products, food and nutrition supplements and medical care.

The properties and applications of chitosan are strongly linked to its morphology, structure and size and these are directly related to the process used for obtaining chitosan. For reasons of clarity, the chitosan obtained as the initial product from chitin will be referred to herein as primary chitosan and the chitosan obtained from the subsequent treatment of this primary chitosan will be referred as modified chitosan.

Traditional primary and modified chitosan have a limited solubility. Chitosan is usually only soluble in acidic medium, typically in a pH range from 1 to 5; this limits its applications. The literature recognises that the solubility of chitosan can be improved by acetylation or chemical modification (“Chemical modification of chitin and chitosan 2: preparation and water soluble property of N-acetylated or N-alkylated partially deacetylated chitins” H. Sashiwa and Y. Shigemasa, Carbohydrate Polymers 39 (1999) 127-138).

The paper “A simple preparation of half N-acetylated chitosan highly soluble in water and aqueous organic solvents” N. Kubota et al. Carbohydrate Research 324 (2000) 268-274, also acknowledges an increase in chitin and chitosan solubility by controlling the degree of substitution. In this paper half-N acetylated chitosan was prepared by degrading chitosan by treatment with NaBO3 and then N-acetylating with acetic anhydride in aqueous acetic acid. The reaction took place in excess of acetic anhydride and was stopped with NaOH. The reaction mixture was dialyzed and the acetylated chitosan was then treated with methanolic KOH for 5 hours and repeatedly washed with MeOH using a centrifuge. Finally, it was dissolved in water.

In H. Sashiwa and Y. Shigemasa's paper chitosan is also acetylated by using an excess of acetyl chloride and stopping the reaction with ice, followed by dialysis and neutralization with NaHCO3.

The acetylation methods proposed in the literature require a large amount of reactants and separation steps which are too cumbersome for use in commercial processes. The purpose of this invention is to provide a simplified method for acetylating chitosan which can be readily implemented at industrial scale.

### SUMMARY OF THE INVENTION

The present invention provides a process for acetylating chitosan. The term chitosan as used herein not only includes the natural polysaccharide β-1,4-poly-D-glucosamine obtained by deacetylation of chitin or by direct isolation from fungi but also includes synthetically produced β-1,4-poly-D-glucosamines and derivatives thereof of equivalent structure to chitosan. By “process for acetylating chitosan” (sometimes also referred herein as “acetylation of chitosan”) is understood a process resulting in an increase of the number of acetyl groups in the chitosan N-groups with respect to the initial chitosan. A molecule of chitosan has two kinds of acetylatable groups, the N-groups and O-groups. The present invention relates to acetylation of the N-groups, i.e, N-acetylation, although it would be understood that a certain amount of the O-acetylation, may occur. O-acetylation in the process of the invention is considered negligible or minor in comparison with N-acetylation.

The first step in the process of the invention is the addition of chitosan to an aqueous acid to form an aqueous chitosan solution or dispersion (referred to herein generally as the “chitosan solution”). An acetylating agent is then added to the aqueous acidic chitosan solution, preferably the acetylating agent being added once the chitosan is well dispersed. The acetylating agent is added to the chitosan solution in a sub-stoichiometric molar amount with respect to the chitosan N-groups to achieve the desired degree of acetylation, ie the chitosan is present in molar excess of the acetylating agent. The amount of acetylating agent required can be theoretically calculated knowing the molecular weight and the N-acetylation degree of the starting chitosan and the desired acetylation degree of the final modified chitosan.

The molecular weight and N-acetylation degree of the starting chitosan can be known either from manufacturer specifications or can be determined by analytical techniques. The molecular weight can be determined by size exclusion chromatography with multiple angle laser light scattering using pullulan as standards and the acetylation degree using the method published in the “Chitin Handbook” (Muzzarelli et. al, ISBN 88-86889-01-1), pages 109-114. This method uses first derivative ultraviolet spectrophotometry to quantify the acetylation degree versus a calibration curve of N-acetyl-D-glucosamine. Details of the method are given herein below.

The theoretical number of moles of acetylating agent (M_{Act}) required to achieve a certain degree of acetylation (DA_{final}) is calculated by:
1) determining the initial degree of N-acetylation (DAᵢₙᵢₜᵢₐₗ) of the chitosan and the total number of N groups in the given weight of chitosan (M_{N});
2) calculating the amount of available amine groups (Av_{N}=M_{Act}) as the difference of the initial degree of N-acetylation minus the required final degree of acetylation multiplied by the number of amine groups to be acetylated: Av_{N}=(DA_{final}−DAᵢₙᵢₜᵢₐₗ)×M_{N}; and
3) the number of moles can be converted into grams of acetylating agent by multiplying by the molecular weight.

Alternatively, a calibration curve can be constructed which correlates the amount of acetylating agent to the acetylation degree of the chitosan, under given reaction conditions. This curve permits easily to determine the amount of acetylating agent required to achieve a determined degree of acetylation.

The amount of acetylating agent required can be determined indirectly by means of the solubility of chitosan. For a chitosan of given molecular weight the solubility at a determined pH is governed by the degree of acetylation. The higher the acetylation degree the higher the pH at which the chitosan dissolves. A calibration curve can be constructed correlating the acetylation degree with solubility at certain pH.

A great advantage associated to the process of the invention is that the resulting chitosan is in an aqueous solution which can be directly used or processed as such or if a more concentrated solution is required some of the water can be evaporated avoiding the need of complex separation processes and the handling of large volume of dangerous solvents. This makes the present process ideal for use at commercial scale.

The acid used to prepare the chitosan solution is preferably selected from acetic, maleic, citric, lactic, salicylic, hydrochloric acid and mixtures thereof. Preferred for use herein is acetic acid for its buffer capacity that avoids a great drop in pH. The concentration of chitosan in the dilute acidic solution is preferably from about 0.1% to about 10%, more preferably from about 0.5% to about 3% by weight, these values being preferred from the process viewpoint, in order to provide a solution with an easily processable rheology. The pH is preferably in the range from about 1 to about 5. The reactants are allowed to react until there is substantially no acetylating agent left, ie, at least about 90%, preferably at least about 95% of the acetylating agent has been consumed in acetylating the chitosan (referred to herein as substantial completion of the acetylation reaction).

The acetylation reaction takes place in aqueous medium. This simplifies the process from the safety and environmental point of view, ie, there is no need to deal with hazardous solvents, which reduces the costs considerably.

In preferred embodiments the acetylating agent is acetic anhydride. Other acetylating agents suitable for use herein include acetyl halides, in particular acetyl chloride.

Some acetylating agents, in particular acetic anhydride, tend to be unstable in aqueous environment due to hydrolysis thereby reducing the number of acetyl groups available for acetylating chitosan. It has now been found that this can be ameliorated by adding a hydrolysis reducing agent to the chitosan solution, preferably before addition of the acetylating agent. This helps to drive the acetylation reaction versus the hydrolysis reaction. It has also been found that the addition of the hydrolysis reducing agent improves the reproducibility of the reaction in terms of the final degree of acetylation of the chitosan produced.

In preferred embodiments the hydrolysis reducing agent is an alcohol, more preferably a low molecular alcohol (ie C1-C6), including methanol, ethanol, propanol, butanol, pentanol, hexanol and mixtures thereof.

In preferred embodiments the chitosan has a molecular weight of from about 50,000 to about 500,000 Da, preferably from about 100,000 to about 300,000 Da. In preferred embodiments the degree of acetylation of the initial chitosan is from about 0 to about 30%, preferably from about 10% to about 25%.

In preferred embodiments the degree of acetylation of the final chitosan is from about 30% to about 80%, more preferably from about 40 to about 70% and even more preferably from about 42 to about 52%.

The process of the invention can produce a range of chitosan products that are soluble at different pHs, broadening the number of potential applications of chitosan. The chitosan products are particularly valuable in the manufacture of pH-triggered water-soluble films, coatings and polymeric substrates, for applications in a variety of products. “pH-triggered” means that the films or substrates are soluble in aqueous media only under certain pH conditions and are otherwise insoluble in the aqueous media. One of these applications includes the use of chitosan for detergent applications, in particular for the manufacture of detergent products in unit dose form. The products can be designed for dissolving at determined pH during the cleaning process.

According to a product aspect, there is provided a unit dose detergent product comprising an enveloping material and a cleaning composition contained therein, wherein the enveloping material comprises acetylated chitosan obtainable or obtained according to the process of the invention. In a preferred embodiment, the unit dose product comprises two or more compartments and at least one of them is prepared from enveloping material comprising chitosan obtainable according to the process of the invention. This permits the dissolution of different compartments at different pHs. For example, if the product is used for automatic dishwashing, one compartment can dissolve during the main wash cycle and the other compartment during the rinse.

Preferred enveloping material for use in at least one of the compartments of a product comprising two or more compartments is chitosan having a degree of acetylation of from about 42 to about 52%. This chitosan is soluble at a pH of from about 8.5 to about 9.5, thereby delaying the dissolution of the enveloping material during the main wash and allowing dissolution during the rinse.

The unit dose detergent product can be in the form of a tablet, pouch, sachet, capsule or the like. Pouches are preferred herein and in particular multi-compartments, especially dual-compartment pouches. Preferred uses of the unit dose detergent product of the invention are laundry and automatic dishwashing, in particular automatic dishwashing.

According to another aspect of the invention, there is provided the use of the acetylated chitosan obtainable or obtained according to the invention as a controlled release agent in detergent products. As explained above the degree of N-acetylation of chitosan determines the pH at which the chitosan is soluble in aqueous solution. Chitosan having a determined degree of acetylation can be used to coat, encapsulate or mix with detergent components or detergents in order to release those components or detergents at the desired pH and to inhibit or prevent release at other pHs.

### DETAILED DESCRIPTION OF THE INVENTION

The present invention envisages a process for acetylating, in particular for N-acetylating, chitosan. The required degree of acetylation is determined a priori and the amount of acetylating agent required to achieve this degree of acetylation is added accordingly. Unit dose detergent packages comprising chitosan obtainable or obtained according to the process of the invention and the use of the acetylated chitosan as a controlled release agent for detergent products are also envisaged by this invention. The pH at which the product dissolves is determined by the degree of acetylation of the chitosan.

The starting chitosan material can be any commercially available chitosan. Suitable chitosan sources may be those derived from shellfish, insects or may be fungally derived. Preferred for use herein are chitosan materials having a molecular weight from about 10,000 to about 500,000 Da.

The first step of the process of the invention is the introduction of chitosan into an aqueous acidic solution. Once the chitosan is added, usually in powder form, the solution should be stirred or otherwise mixed in order to disperse the chitosan into the solution and achieve good wetting of the powder. Solubilisation of the powder is not essential, good dispersion of the powder is usually enough at this stage.

The acid used in this first step is preferably selected from acetic, maleic, citric, lactic, salicylic, hydrochloric acid and mixtures thereof. Preferred for use herein is acetic acid. The concentration of chitosan in the dilute acidic solution is preferably from about 0.1% to about 10%, more preferably from about 0.5% to about 2% by weight, these values being preferred from the process viewpoint, in order to provide a solution with the right consistency and easy to process. The pH is preferably in the range from about 1 to about 5. Crystallization inhibitors, as for example diethylene triamine penta(methylene phosphonic) acid, can be added to the solution to avoid premature seed formation which can become crystal growth centres. The resulting acidic chitosan solution can be optionally filtered to remove insoluble impurities.

The reaction, i.e, steps a), b) and c) can take place in a single reactor or step a) can take place in a first reactor and step b) and c) in a second reactor. Any stirred reactor can be used for the purpose of this invention. The process can be carried out in continuous or batch manner. The process is preferably carried out at ambient temperature (ie, about 23° C.) and atmospheric pressure. The temperature and/or pressure of reaction can be increased in order to reduce the residence time.

Determination of the Acetylation Degree

Three solutions of acetic acid about 0.01, 0.02 and 0.003 M are prepared and the first derivative spectra from 240 to 190 nm, against water, are recorded. The superposition of the three spectra shows the zero crossing point for the acid.

Four or five reference solutions of N-acetylglucosamine in the range 0.5-3.5 mg in 100 ml of 0.01 M acetic acid are prepared and the spectra are recorded as before.

All spectra recorded are superposed and the height H (mm), for each reference concentration above the zero crossing point, is measured. A calibration curve (H versus concentration of N-acetylglucosamine) is drawn. The curve equation H=f(C) is determined.

500 mg of dry chitosan (i.e. previously freeze dried) are dissolved in 50 ml of 0.1 M acetic acid and then diluted to 500 ml with water. In case the degree of acetylation is high, a further 10-fold dilution is necessary.

The solution is transferred to a Far-UV cuvette with 10 mm path length.

Different spectrophotometers can be used: for instance the Beckman DU 640, the Kontron Uvikov 810 and the Perkin Elmer 550 SE. The derived spectra are obtained at a light with of 1 nm, a scanning speed of 30 nm/min and a time constant of 4 sec, chart speed 10 cm/min.

For degree of acetylation lower than 0.11 the final result should be corrected with a coefficient deduced from the correction curve.

Enveloping Material

The enveloping material may further comprise additional polymeric materials. Preferred polymers, copolymers or derivatives thereof suitable for use as polymeric material are selected from polyvinyl alcohols (PVA), polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferred polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and most preferably selected from polyvinyl alcohols, polyvinyl alcohol copolymers and hydroxypropyl methyl cellulose (HPMC), and combinations thereof.

Mixtures of polymers can be beneficial to further control the mechanical and/or dissolution properties of the enveloping material, depending on the application thereof and the required needs. Suitable mixtures include for example mixtures wherein one polymer has a higher water-solubility than another polymer, and/or one polymer has a higher mechanical strength than another polymer. Also suitable are mixtures of polymers having different average weight molecular weights.

Most preferred polymeric material is PVA having an average weight molecular weight, preferably from 1,000 Da to 1,000,000 Da, more preferably from 10,000 Da to 300,000 Da, and most preferably from 20,000 Da to 150,000 Da, such as those known under the trade reference Monosol M8630, as sold by Chris-Craft Industrial Products of Gary, Indiana, US.

When such polymeric materials, in particular PVA, are further comprised in the enveloping material together with the acetylated chitosan, it is preferred that the weight ratio of the chitosan to the additional polymeric material be from 0.1:100 to 50:100, preferably from 1:100 to 10:100.

The enveloping material herein can also comprise one or more additive ingredients. For example, it can be beneficial to add plasticisers, for example glycerol, ethylene glycol, diethyleneglycol, propylene glycol, sorbitol and mixtures thereof. Other additives include functional detergent additives to be delivered to the wash water, for example organic polymeric dispersants, etc.

The enveloping material could be in the form of a film, coating, injection moulding substrate or the like. Preferably, the enveloping material is used to coat solid bodies, such as detergent tablets or other inserts used in detergents, such as balls, noodles, discs, etc. The inserts can be used on their own or as part of detergent products.

The enveloping material can be sprayed on detergent, in powder or compacted form (i.e., tablets and other solid bodies).

A process for making a cleaning composition comprising the N-acetylated chitosan obtained according to the process of the invention comprises the steps of:
(a) preparing a cleaning composition;
(b) enveloping a quantity of the cleaning composition for example by coating, spraying, encapsulation, pouch formation, injection moulding, etc with a film or composition comprising the N-acetylated chitosan.

A cleaning composition may be coated using conventional procedures such as those known for tablet coatings. The N-acetylated chitosan of the present invention can be sprayed onto the composition from a melt or from a solution or dispersion. In this case, the composition to be coated is situated on a fluid bed or in a tablet coating pan. The composition to be coated may also be dispersed in molten N-acetylated chitosan in order then to be processed to form granulated material by spraying. For this purpose known spray-cooling, spray freezing or rotating disc procedures can be used. The N-acetylated chitosan layer can be applied from an aqueous solvent or another solvent with the aid of spray-coating. The composition to be coated is contained in this case in a tablet coating pan or on a fluid bed. It is also possible to disperse the cleaning composition to be coated in the solution with amino-acetylated polysaccharide and then to spray-dry the dispersion. Alternatively, the N-acetylated chitosan may be applied by coacervation technique.

Preferably, the unit dose detergent products according to the invention are in the form of a pouch. The pouch can be made according to the processes described in WO 02/42408.

Cleaning Composition

The cleaning compositions herein can comprise traditional detergency components and can also comprise organic solvents having a cleaning function and organic solvents having a carrier or diluent function or some other specialised function. The compositions will generally be built and comprise one or more detergent active components which may be selected from bleaching agents, surfactants, alkalinity sources, enzymes, thickeners (in the case of liquid, paste, cream or gel compositions), anti-corrosion agents (e.g. sodium silicate) and disrupting and binding agents (in the case of powder, granules or tablets). Highly preferred detergent components include a builder compound, an alkalinity source, a surfactant, an enzyme and a bleaching agent.

Surfactant

In the detergent product of the present invention the detergent surfactant is preferably low foaming by itself or in combination with other components (i.e. suds suppressers). Surfactants suitable herein include anionic surfactants such as alkyl sulfates, alkyl ether sulfates, alkyl benzene sulfonates, alkyl glyceryl sulfonates, alkyl and alkenyl sulphonates, alkyl ethoxy carboxylates, N-acyl sarcosinates, N-acyl taurates and alkyl succinates and sulfosuccinates, wherein the alkyl, alkenyl or acyl moiety is C₅-C₂₀, preferably C₁₀-C₁₈ linear or branched; cationic surfactants such as chlorine esters (U.S. Pat. No. 4,228,042, U.S. Pat. No. 4,239,660 and U.S. Pat. No. 4,260,529) and mono C₆-C₁₆ N-alkyl or alkenyl ammonium surfactants wherein the remaining N positions are substituted by methyl, hydroxyethyl or hydroxypropyl groups; low and high cloud point nonionic surfactants and mixtures thereof including nonionic alkoxylated surfactants (especially ethoxylates derived from C₆-C₁₈ primary alcohols), ethoxylated-propoxylated alcohols (e.g., BASF Poly-Tergent® SLF18), epoxy-capped poly(oxyalkylated) alcohols (e.g., BASF Poly-Tergent® SLF18B—see WO-A-94/22800), ether-capped poly(oxyalkylated) alcohol surfactants, and block polyoxyethylene-polyoxypropylene polymeric compounds such as PLURONIC®, REVERSED PLURONIC®, and TETRONIC® by the BASF-Wyandotte Corp., Wyandotte, Mich.; amphoteric surfactants such as the C₁₂-C₂₀ alkyl amine oxides (preferred amine oxides for use herein include C₁₂ lauryldimethyl amine oxide, C₁₄ and C₁₆ hexadecyl dimethyl amine oxide), and alkyl amphocarboxylic surfactants such as Miranol™ C2M; and zwitterionic surfactants such as the betaines and sultaines; and mixtures thereof. Surfactants suitable herein are disclosed, for example, in U.S. Pat. No. 3,929,678 , U.S. Pat. No. 4,259,217, EP-A-0414 549, WO-A-93/08876 and WO-A-93/08874. Surfactants are typically present at a level of from about 0.2% to about 30% by weight, more preferably from about 0.5% to about 10% by weight, most preferably from about 1% to about 5% by weight of composition. Preferred surfactant for use herein are low foaming and include low cloud point nonionic surfactants and mixtures of higher foaming surfactants with low cloud point nonionic surfactants which act as suds suppresser therefor.

Builder

Builders suitable for use in cleaning compositions herein include water-soluble builders such as citrates, carbonates and polyphosphates e.g. sodium tripolyphosphate and sodium tripolyphosphate hexahydrate, potassium tripolyphosphate and mixed sodium and potassium tripolyphosphate salts; and partially water-soluble or insoluble builders such as crystalline layered silicates (EP-A-0164514 and EP-A-0293640) and aluminosilicates inclusive of Zeolites A, B, P, X, HS and MAP. The builder is typically present at a level of from about 1% to about 80% by weight, preferably from about 10% to about 70% by weight, most preferably from about 20% to about 60% by weight of composition.

Amorphous sodium silicates having an SiO₂:Na₂O ratio of from 1.8 to 3.0, preferably from 1.8 to 2.4, most preferably 2.0 can also be used herein although highly preferred from the viewpoint of long term storage stability are compositions containing less than about 22%, preferably less than about 15% total (amorphous and crystalline) silicate.

Enzyme

Enzymes suitable herein include bacterial and fungal cellulases such as Carezyme and Celluzyme (Novo Nordisk A/S); peroxidases; lipases such as Amano-P (Amano Pharmaceutical Co.), M1 Lipase^{R} and Lipomax^{R} (Gist-Brocades) and Lipolase^{R} and Lipolase Ultra^{R} (Novo); cutinases; proteases such as Esperase^{R}, Alcalase^{R}, Durazym^{R} and Savinase^{R} (Novo) and Maxatase^{R}, Maxacal^{R}, Properase^{R} and Maxapem^{R} (Gist-Brocades); and amylases such as Purafect Ox Am^{R} (Genencor) and Termamyl^{R}, Ban^{R}, Fungamyl^{R}, Duramyl^{R}, and Natalase^{R} (Novo); pectinases; and mixtures thereof. Enzymes are preferably added herein as prills, granulates, or cogranulates at levels typically in the range from about 0.0001% to about 2% pure enzyme by weight of composition.

Bleaching Agent

Bleaching agents suitable herein include chlorine and oxygen bleaches, especially inorganic perhydrate salts such as sodium perborate mono-and tetrahydrates and sodium percarbonate optionally coated to provide controlled rate of release (see, for example, GB-A-1466799 on sulfate/carbonate coatings), preformed organic peroxyacids and mixtures thereof with organic peroxyacid bleach precursors and/or transition metal-containing bleach catalysts (especially manganese or cobalt). Inorganic perhydrate salts are typically incorporated at levels in the range from about 1% to about 40% by weight, preferably from about 2% to about 30% by weight and more preferably from abut 5% to about 25% by weight of composition. Peroxyacid bleach precursors preferred for use herein include precursors of perbenzoic acid and substituted perbenzoic acid; cationic peroxyacid precursors; peracetic acid precursors such as TAED, sodium acetoxybenzene sulfonate and pentaacetylglucose; pemonanoic acid precursors such as sodium 3,5,5-trimethylhexanoyloxybenzene sulfonate (iso-NOBS) and sodium nonanoyloxybenzene sulfonate (NOBS); amide substituted alkyl peroxyacid precursors (EP-A-0170386); and benzoxazin peroxyacid precursors (EP-A-0332294 and EP-A-0482807). Bleach precursors are typically incorporated at levels in the range from about 0.5% to about 25%, preferably from about 1% to about 10% by weight of composition while the preformed organic peroxyacids themselves are typically incorporated at levels in the range from 0.5% to 25% by weight, more preferably from 1% to 10% by weight of composition. Bleach catalysts preferred for use herein include the manganese triazacyclononane and related complexes (U.S. Pat. No. 4,246,612, U.S. Pat. No. 5,227,084); Co, Cu, Mn and Fe bispyridylamine and related complexes (U.S. Pat. No. 5,114,611); and pentamine acetate cobalt(III) and related complexes (U.S. Pat. No. 4,810,410).

Low Cloud Point Non-Ionic Surfactants and Suds Suppressers

The suds suppressers suitable for use herein include nonionic surfactants having a low cloud point. “Cloud point”, as used herein, is a well known property of nonionic surfactants which is the result of the surfactant becoming less soluble with increasing temperature, the temperature at which the appearance of a second phase is observable is referred to as the “cloud point” (See Kirk Othmer, pp. 360-362). As used herein, a “low cloud point” nonionic surfactant is defined as a nonionic surfactant system ingredient having a cloud point of less than 30° C., preferably less than about 20° C., and even more preferably less than about 10° C., and most preferably less than about 7.5° C. Typical low cloud point nonionic surfactants include nonionic alkoxylated surfactants, especially ethoxylates derived from primary alcohol, and polyoxypropylene/polyoxyethylene/polyoxypropylene (PO/EO/PO) reverse block polymers. Also, such low cloud point nonionic surfactants include, for example, ethoxylated-propoxylated alcohol (e.g., BASF Poly-Tergent® SLF18) and epoxy-capped poly(oxyalkylated) alcohols (e.g., BASF Poly-Tergent® SLF18B series of nonionics, as described, for example, in U.S. Pat. No. 5,576,281).

Preferred low cloud point surfactants are the ether-capped poly(oxyalkylated) suds suppresser having the formula:

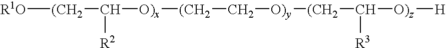

wherein R¹ is a linear, alkyl hydrocarbon having an average of from about 7 to about 12 carbon atoms, R² is a linear, alkyl hydrocarbon of about 1 to about 4 carbon atoms, R³ is a linear, alkyl hydrocarbon of about 1 to about 4 carbon atoms, x is an integer of about 1 to about 6, y is an integer of about 4 to about 15, and z is an integer of about 4 to about 25.

Other low cloud point nonionic surfactants are the ether-capped poly(oxyalkylated) having the formula:

R_{I}O(R_{II}O)ₙCH(CH₃)OR_{III}

wherein, R_{I} is selected from the group consisting of linear or branched, saturated or unsaturated, substituted or unsubstituted, aliphatic or aromatic hydrocarbon radicals having from about 7 to about 12 carbon atoms; R_{II} may be the same or different, and is independently selected from the group consisting of branched or linear C₂ to C₇ alkylene in any given molecule; n is a number from 1 to about 30; and R_{III} is selected from the group consisting of:
(i) a 4 to 8 membered substituted, or unsubstituted heterocyclic ring containing from 1 to 3 hetero atoms; and
(ii) linear or branched, saturated or unsaturated, substituted or unsubstituted, cyclic or acyclic, aliphatic or aromatic hydrocarbon radicals having from about 1 to about 30 carbon atoms;
(b) provided that when R² is (ii) then either: (A) at least one of R¹ is other than C₂ to C₃ alkylene; or (B) R² has from 6 to 30 carbon atoms, and with the further proviso that when R² has from 8 to 18 carbon atoms, R is other than C₁ to C₅ alkyl.

Other suitable components herein include organic polymers having dispersant, anti-redeposition, soil release or other detergency properties invention in levels of from about 0.1% to about 30%, preferably from about 0.5% to about 15%, most preferably from about 1% to about 10% by weight of composition. Preferred anti-redeposition polymers herein include acrylic acid containing polymers such as Sokalan PA30, PA20, PA15, PA10 and Sokalan CP10 (BASF GmbH), Acusol 45N, 480N, 460N (Rohm and Haas), acrylic acid/maleic acid copolymers such as Sokalan CP5 and acrylic/methacrylic copolymers. Preferred soil release polymers herein include alkyl and hydroxyalkyl celluloses (U.S. Pat. No. 4,000,093), polyoxyethylenes, polyoxypropylenes and copolymers thereof, and nonionic and anionic polymers based on terephthalate esters of ethylene glycol, propylene glycol and mixtures thereof.

Heavy metal sequestrants and crystal growth inhibitors are suitable for use herein in levels generally from about 0.005% to about 20%, preferably from about 0.1% to about 10%, more preferably from about 0.25% to about 7.5% and most preferably from about 0.5% to about 5% by weight of composition, for example diethylenetriamine penta(methylene phosphonate), ethylenediamine tetra(methylene phosphonate)hexamethylenediamine tetra(methylene phosphonate), ethylene diphosphonate, hydroxy-ethylene-1,1-diphosphonate, nitrilotriacetate, ethylenediaminotetracetate, ethylenediamine-N,N′-disuccinate in their salt and free acid forms.

The compositions herein can contain a corrosion inhibitor such as organic silver coating agents in levels of from about 0.05% to about 10%, preferably from about 0.1% to about 5% by weight of composition (especially paraffins such as Winog 70 sold by Wintershall, Salzbergen, Germany), nitrogen-containing corrosion inhibitor compounds (for example benzotriazole and benzimadazole—see GB-A-1137741) and Mn(II) compounds, particularly Mn(II) salts of organic ligands in levels of from about 0.005% to about 5%, preferably from about 0.01% to about 1%, more preferably from about 0.02% to about 0.4% by weight of the composition.

Other suitable components herein include colorants, water-soluble bismuth compounds such as bismuth acetate and bismuth citrate at levels of from about 0.01% to about 5%, enzyme stabilizers such as calcium ion, boric acid, propylene glycol and chlorine bleach scavengers at levels of from about 0.01% to about 6%, lime soap dispersants (see WO-A-93/08877), suds suppressors (see WO-93/08876 and EP-A-0705324), polymeric dye transfer inhibiting agents, optical brighteners, perfumes, fillers and clay.

Liquid detergent compositions can contain low quantities of low molecular weight primary or secondary alcohols such as methanol, ethanol, propanol and isopropanol. Other suitable carrier solvents suitable herein include glycerol, propylene glycol, ethylene glycol, 1,2-propanediol, sorbitol and mixtures thereof.

### EXAMPLE 1

2 g of chitosan, Chitoclear ex Primex having an approximate molecular weight of 135,300 DA and an acetylation degree of approximately 17%, was transferred to a 250 ml 3-necked round bottom flask, containing a stirrer bead. 90 ml of deionized water was added to the flask with stirring. 0.53 g of acetic acid was dissolved in 5 g of deionized water and added in one portion to the reaction mixture, whilst stirring vigorously. The stirring continued for about 15 hours. Thereafter, 4 g of ethanol was added to the reaction and the mixture stirred for about 2 hours.

0.35 g of acetic anhydride was dissolved in 1 g of ethanol and added drop-wise to the reaction whilst stirring vigorously. After one hour a solution comprising chitosan having a molecular weight of about 178,000 Da and a degree of acetylation of 48% was obtained. The temperature of the reaction mixture prior to and during the addition was 23° C.

### EXAMPLE 2

A solution obtained as described in example 1 is used to make a chitosan film by casting the film on an A4 glass sheet to the required thickness, for example between 0.03 and 0.06 inches. The film is left to dry at room temperature overnight.

### EXAMPLE 3

A solution obtained as described in example 1 is used to make a PVA/chitosan film as follows:

1) Dissolve 20 g of PVA in 100 g of deionised water, and then mix in 100 g of Chitosan solution (2%).

2) Cast the solution on glass or A4 plastic sheet to the required thickness. Film cast at 0.03 to 0.06 inches.

3) Allow the film to dry at room temperature overnight.

The films obtained as described in examples 2 and 3, can be used to make dual compartment film as follows: placing PVA film into a mould, introducing a first cleaning composition, placing a second film obtained according to examples 2 or 3, introducing a second cleaning composition, placing a third film obtained according to examples 2 or 3 and sealing by means of heat or solvent sealing.

The composition of example 1 can be used to coat inserts, that can be placed in pouches or tablets.

1. A process for making modified N-acetylated chitosan comprising the steps of:
a) forming an aqueous solution of chitosan in aqueous acid;
b) mixing the resulting solution of step a) with an acetylating agent in a chitosan:acetylating agent sub-stoichiometric amount; and
c) allowing the chitosan and acetylating agent to react for a length of time sufficient for substantial completion an acetylation reaction.

2. A process according to claim 1 wherein the solution of step a) comprises an acid selected from acetic, maleic, citric, lactic, salicylic, hydrochloric acid and mixtures thereof.

3. A process according to claim 1 wherein the concentration of chitosan in the solution of step a) is from about 0.1% to about 5% by weight and the pH is in the range from about 1 to about 5.

4. A process according to claim 1 wherein the acetylating agent is acetic anhydride.

5. A process according to claim 1 wherein the acetylation reaction takes place in the presence of about 0.1 to 10% by weight of the chitosan solution of a hydrolysis reducing agent.

6. A process according to claim 5 wherein the hydrolysis reducing agent is an alcohol.

7. A process according to claim 1 wherein the chitosan has a molecular weight of from about 50,000 to about 500,000 Da.

8. A process according to claim 1 wherein the degree of acetylation of the starting chitosan added in step a) is from about 0 to about 30%.

9. A process according to claim 1 wherein the degree of acetylation of the modified N-acetylated chitosan is from about 40% to about 80%.

10. A unit dose detergent product comprising an enveloping material and a cleaning composition contained therein, wherein the enveloping material comprises modified N-acetylated chitosan obtainable according to any of the preceding claims.

11. A unit dose detergent according to claim 10 wherein the package comprises two or more compartments, at least one compartment being prepared from enveloping material comprising chitosan obtainable according to claim 1.

12. A unit dose detergent according to claim 10 wherein the degree of acetylation of the chitosan is from about 40 to about 80.

13. A pH-triggered water-soluble film or polymeric substrate comprising a modified N-acetylated chitosan obtainable according to the process of claim 1.
